(19) United States
(12) Patent Application Publication
(10) Pub. No.: US 2026/0108207 A1
(43) Pub. Date: Apr. 23, 2026

(54) APPARATUS AND METHOD FOR CALCULATING AN OPTIMUM MEDICATION DOSE

(71) Applicants: Nazmi Peyman, Richmond, VA (US); Edmond Zahedi, Richmond, VA (US)

(72) Inventors: Nazmi Peyman, Richmond, VA (US); Edmond Zahedi, Richmond, VA (US)

(21) Appl. No.: 19/420,103

(22) Filed: Dec. 15, 2025

Related U.S. Application Data
(63) Continuation-in-part of application No. 17/534,262, filed on Nov. 23, 2021, now Pat. No. 12,518,860.

(51) Int. Cl. A61B 5/00; A61J 7/00; G16H 20/13

(52) U.S. Cl. CPC A61B 5/4839; A61B 5/7267; A61B 5/7465; A61J 7/0076; G16H 20/13

(57) ABSTRACT

An apparatus and method for calculating an optimum medication dose for a patient based on a multiplicity of factors that can affect the medical condition and thus the medication dose. The apparatus includes a trained machine learning based model that can calculate an optimum medication dose based on medication data, diagnostic data, biodata, and plurality of parameters affecting the medical condition. The apparatus can also include a dispenser for dispensing the calculated medication dose.

### CROSS REFERENCE TO RELATED APPLICATIONS

This Application is continuation-in-part of a U.S. patent application Ser. No. 17/534,262, filed on Nov. 23, 2021, which is incorporated herein by reference in its entirety.

### FIELD OF INVENTION

The invention relates to determining and dispensing an optimum medication dose, and more particularly, to systems integrating wearable devices, advanced artificial intelligence models, and expanded delivery methods for personalized dosing.

### BACKGROUND

When prescribing medication, a medical professional determines the medication dose for a patient. The term “medication dose” refers to the amount of medicine to be administered or ingested at one time. Achieving the correct dose is critical: excess dosing may cause adverse side effects, whereas insufficient dosing may lead to sub-optimal therapeutic outcomes. The optimal medication dose can depend on multiple factors, which may vary from one patient to another and may also change over time. For a given patient, dosage requirements may be influenced by genetic, environmental, and psychological factors, including but not limited to anxiety levels, lifestyle, dietary habits, concurrent prescribed or non-prescribed drugs, and exposure to airborne or waterborne chemicals. Seasonal effects may also alter disease severity and the corresponding therapeutic requirement. For example, an individual's blood pressure may fluctuate due to a combination of personal and environmental variables.

In addition to the direct pharmacological effect of a medication on a patient, numerous environmental, social, and external influences can alter the patient's physiological response. Examples include changes in employment status, family relationships, significant life events, relocation or travel, and even week-to-week routine cycles.

Furthermore, the interaction between multiple medications must be considered in determining the appropriate dose for a medical condition such as hypertension. Currently, no models exist that comprehensively incorporate such drug interactions while accounting for environmental and external influences, including factors such as employment changes, personal events, relocation, travel, and cyclical behavioral patterns.

Existing systems for calculating medication dosages also suffer from limitations in diagnostic and biodata integration. Many lack seamless connectivity with consumer wearable devices, robust data provenance mechanisms, and support for multi-drug or multi-route delivery systems.

Accordingly, there is a need for systems capable of calculating individualized, continuously optimized medication doses based on a set of directly or indirectly recorded physiological and contextual values, rather than relying on nominal or discrete dosage units provided by conventional medication forms. Such systems would enable dispensing of an optimally computed medication amount tailored to current patient-specific conditions. In particular, there is a need for solutions that extend beyond hypertension and oral dosing to support chronic conditions such as diabetes, asthma, and sleep disorders.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

A principal object of the present invention is to provide an apparatus and method for determining an optimum medication dose for a medical condition in a patient.

Another object of the present invention is to enable calculation of the medication dose based on multiple patient-specific factors, including personal, physiological, and environmental factors.

A further object of the present invention is to allow periodic or continuous optimization of the medication dose in accordance with changes in the multiple factors over time.

Yet another object of the present invention is to provide determination of the optimum medication dose for hypertension by measuring, directly or indirectly, the plurality of factors affecting blood pressure and monitoring variations therein.

Still another object of the present invention is to dispense or facilitate dispensing of the calculated optimum amount of medication to the patient.

In one aspect disclosed is an apparatus and method for calculating an optimum medication dose for a patient based on a multiplicity of factors. The disclosed apparatus can receive as an input the prescription of the patient, diagnostic data, medical histories of the patient and his family members such as parents and siblings, and biodata of the patient. The biodata can include information about the lifestyle of the patient, planned life activities, gender, age, and genetic data. The disclosed apparatus can also monitor the activities of the patient, namely energy expenditure and the environment of the patient for determining the value of multiple parameters that can affect the medical condition and thus the medication dose. Based on such data, the apparatus can generate a model tailored to the physiological profile of the patient wherein this model can be used to calculate the optimum medication dose.

Herein, the definition of optimum is the minimum dose of medication that allows maintaining the blood pressure of the patients within predetermined range values. The predetermined range for blood pressure can vary from patient to patient and can be determined by the medical professional. Moreover, in the same patient, the predetermined range for blood pressure can vary with progress in the treatment. For example, the medical professional may decide that for a certain patient, the systolic BP can be within the range of 135-145 mmHg, whereas the diastolic values can be within 85-95 mmHg. As long as the recorded blood pressure values remain within the specified range, the blood pressure control result can be considered “optimum”. It is understood that certain embodiments are directed to calculating optimum medication dose for hypertensive patients are for illustrative purposes only and other medical conditions are within the scope of the present invention

In one aspect, the disclosed apparatus can continue monitoring the different parameters, diagnostic data, effect of the medicine on the patient, and like and based on changes over time, the model can be automatically updated, keeping the dispensed medication dose adjusted to the optimum medication dosage that can produce an optimum therapeutic effect in the patient.

In one aspect, the disclosed apparatus can dispense the calculated medication dose. This medication dose can vary in a much more granular way than what is permitted using pills for example, where the dispensing unit of medication is discrete.

In one aspect, disclosed is a method for calculating an optimum medication dose for a medical condition in a patient, the method implemented within an apparatus, the apparatus comprises a processor and a memory, wherein the method comprises the steps of: training, a machine learning-based medication dose model using at least diagnostic data, medication data, and biodata of a patient to determine a medication dose for a medical condition of the patient; storing the medication dose model in the memory; and processing the medication dose model, by the processor, based on at least current diagnostic data and current medication data of the patient for calculating a first medication dose.

In one implementation of the method, the method further comprises the steps of dispensing, by the apparatus, the calculated first medication dose.

In one implementation of the method, the method further comprises the steps of monitoring the diagnostic data, the medication data, and a plurality of parameters that affects the medical condition of the patient; updating the medication dose model based on changes in the medication data, the diagnostic data, and the plurality of parameters; and processing the updated medication dose model to calculate a second medication dose for the medical condition. The medical condition can be hypertension. The diagnostic data comprises systolic and diastolic blood pressure of the patient. The medication data comprises details of medicines taken by the patient. The biodata comprises genetic information, physiological information, gender, age, medical history, sleep habits, and physical activities. The plurality of parameters comprises quantified genetic information, medical history, quantified employment details, quantified mental state, energy expenditure, calories intake, partial oxygen, and carbon dioxide saturation levels in blood, and sleep patterns.

In one aspect, disclosed is an apparatus for dispensing calculated dose of medicine, sensors to determine values of different parameters affecting the medical condition, and processor to implement the above method.

In one aspect, disclosed is a centralized system that provides wearable device integration. For example, different devices for healthcare including smartwatches, glucose monitors, and inhaler sensors can be integrated. The centralized system is based on advanced artificial intelligence techniques including reinforcement learning and federated learning across distributed datasets. Moreover, for building trust and auditability, the disclosed system may be based on Blockchain or the like database. The disclosed system may be made versatile having wide compatibility with different kinds of healthcare devices, such as inhalers, injectables, and patches. Also, the disclosed system may provide telehealth dashboards providing real-time clinician access.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a block diagram showing an architecture of the system, according to an exemplary embodiment of the present invention.

FIG. 2 is a flowchart illustrating steps in training the model, according to an exemplary embodiment of the present invention.

FIG. 3 is a flowchart illustrating steps of the method for calculating an optimum dose for a patient, according to an exemplary embodiment of the present invention.

FIG. 4 is a block diagram illustrating the medication dose model, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed are an apparatus and method for calculating the medication dose to address hypertension for a patient based on different factors, such as physiological and lifestyle factors that can affect the disease or the medication dose. The disclosed apparatus can also dispense the calculated medication dose by combining small sub-units of the medication in different combinations. The disclosed apparatus and method can also monitor the different parameters for changes or trends over time and change the medication dose if required. In diseases like hypertension wherein multiple factors can contribute to hypertension, the disclosed apparatus can provide better management of hypertension by optimizing the medication dose based on the multiple parameters and closely monitoring the effects of the medicine on the patient. Mental diseases like anxiety and depression which are greatly affected by the lifestyle and mood of a patient can also be managed better by optimizing the medication dose based on the actual needs of the patient. Similarly, lifestyle diseases such as diabetes which require management of glucose levels within predetermined limits could be better managed by optimizing the medication dose calculated based on the trends in blood glucose levels over time and physical activities of the patient.

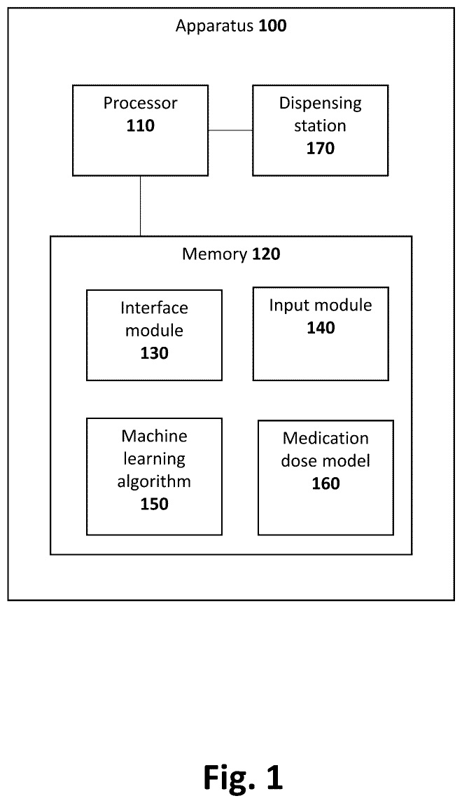

Referring to FIG. 1 which shows a block diagram showing an exemplary embodiment of the disclosed apparatus 100. The apparatus can include a processor 110 which can be a desktop processor or an embedded processor. The functioning of a processor is to logically process information fetched from memory and is known to a skilled person. The apparatus 100 can also include a memory 120 that can be used to store data. The use of memory in computing devices is also known to a skilled person and hence not described here. The memory 120 can include an interface module 130 which upon execution by the processor can provide an interface for interacting with the patient, medical professionals, and/or technicians. The memory 120 may also include an input module 140 that can receive a prescription and other data/parameters that can affect the medical condition and the medication dose. The input module 140 is connected to a variety of sensors allowing the algorithm to have access to the required data to perform the tuning of the medication dose model 160 hence the calculation of the optimal medication dose. The memory 120 can include a machine learning algorithm 150 which upon execution by the processor can process the prescription and the data related to multiple parameters received by the input module 140 to train and update the medication dose model 160. The medication dose model 160 can be stored in the memory 120 and upon execution by the processor can calculate the optimum medication dose for the patient. Also, can be seen in FIG. 1 is a dispensing station 170 that can receive the calculated medication dose through the processor 110 and dispense the medicine in calculated continuous doses.

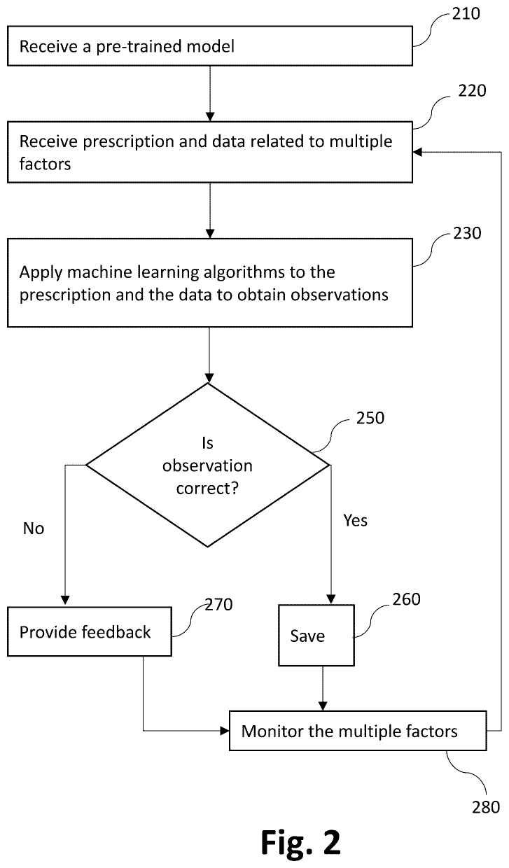

Referring to FIG. 2, which is a flow chart showing steps in training the medication dose model 160. First, a pre-trained model can be received at step 210. The medication dose model 160 can be pre-trained based on the theoretical and technical knowledge, medical data of patients, and optionally using test subjects. Theoretical and technical knowledge can include knowledge about the physiology of the body, diseases, medications, doses of medications, therapeutic doses, toxic dose level, side effects, gender, weight, height, and the like. Historical patient data can also be used in the training of the pre-trained medication dose model 160. The pre-trained model can be further personalized for each patient. The prescription data, diagnostic data, and data related to multiple parameters for a patient can be received at step 220. To adjust the medication dose model for a specific patient, the machine learning algorithms can be applied to the prescription and the data based on the pre-trained model to make observations, at step 230. The observations can also be the determining values of different parameters including medication doses. The observations can also be predicted blood pressure values based on the medication of the patient. The observations can be presented for checking at step 240. If an observation matches the criteria set by the medical professional at step 240, the same can be saved at step 250. Else, if the observation is outside the range specified by the medical professional at step 240, feedback can be provided that can help the machine learning algorithm to further adjust the model (learning), at step 270. For example, the predicted blood pressure can be compared with actual blood pressure. At each iteration, multiple factors are continuously monitored, and new data is created at step 280. Eventually, the medical dose model can be validated when the model can predict the values of the blood pressure over a significant period. This period can vary between a few days to a few weeks. During this time, the model can predict the blood pressure values within the range specified by the medical professional. The model calculates these predictions based on the medication taken by the patient and all other data that was input and recorded during this period.

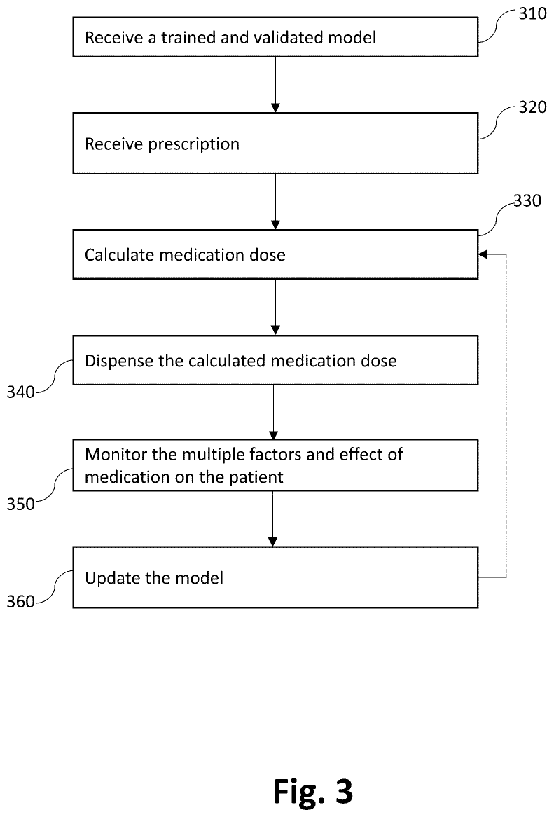

Referring to FIG. 3 which shows steps in calculating the medication dose. Once the medication dose model 160 is trained and validated, it can be approved by the physician for use by the patient, at step 310. The same or new prescription can be received at step 320. Using the medication dose model 160, the medication dose can be calculated by the machine learning algorithms, at step 330. The calculated medication doses can then be dispensed by the dispensing station, at step 340. The apparatus can continue to monitor the multiple factors and the effects of medicine on the patient, at step 350. Based on the newly created data at step 350, the machine learning algorithms can update the medication dose model 160, at step 360 and the updated medication dose model 160 can be used to calculate subsequent medication doses.

In one exemplary embodiment, the multiple factors can include other medications taken by the patient and include prescribed and non-prescribed medications; supplements; herbal medicines and teas, vital signs, and physical activities; lifestyle activities; and the like. Each data point can be time-labeled, and the value of the data can be either accurately determined or approximated. Examples of accurate data can include the doses of medications, values of systolic and diastolic blood pressure, and saturated blood oxygen levels. Examples of approximated data can include physical activities, amount of food ingested, consumed calories, sleep, and stress. Such subjected data can be quantified based on predetermined rules. For example, stress can be quantified on a scale of 1-5, wherein 1 is mild stress and 5 is extreme stress levels.

The machine learning algorithm can use all available time-series data as well as other biodata of the patient to create the data-driven medication dose model 160 which can be validated using data that has not been used for training. The training and validation of the model may take a few days and up to a few weeks so that at least a whole cycle of events can be covered, during which the machine learning algorithm can continuously compare the predicted values of the vital signs with the actual readings, given the current model parameters, the medication consumption, and activities. Once the model has been validated by the results of the predictions of the model being within the range indicated by the healthcare professional, it can be further reviewed by healthcare professionals for their approval.

A series of safety features can be implemented by (1) constantly comparing the desirable vital signs with the actual values, and (2) ensuring that the maximum and minimum values of mediation doses are dispensed so that if any significant discrepancy is noted between the predicted and actual values, the apparatus can alert the patient and request the intervention of a healthcare provider by sending all diagnostic and medication data, to prevent any under or overdose.

In one embodiment, the machine learning algorithm can include Deep Learning, whereas a significant amount of data is recorded to extract meaningful insights. The number and types of neurons in such algorithms are determined based on the complexity of the problem. As there are multiple implementations of such algorithms as part of a more general Artificial Intelligence realm, no further details are provided here.

Algorithms such as deep-learning that takes into consideration the physician's prescribed medications, time-labeled information such as vital signs data, frequently recorded blood pressure values, current and future lifestyle activities, consumed prescribed and non-prescribed medications, and food amount.

In one exemplary embodiment, the apparatus can dispense the calculated medication dose of medicine for the patient. The apparatus can include unit amounts of the medicine in desired physical form such as granules, syrup, and like. For example, 10 mg units of medicine can be present in the form of a granule. The granules can be combined to form a single medication dose for the patient. 300 mg of calculated medication dose can be formed by combining 30 units of 10 mg granules.

In one aspect, different types of sensors can be used to monitor vital signs, behavior, stress levels, sleeping habits, physical activity, environmental factors, and like factors from a patient. A survey type of questions can also be asked from the patient to determine the state of the patient, such as pain, anxiety levels, mood, and any external events affecting the mental state of the user. Sensors included in the form of a band or smartwatches are known that can measure and monitor various physical and psychological parameters and activities of a person in normal day-to-day life. These bands are easy to wear and carry without affecting the daily actives of the people. Also, such bands or smartwatches can monitor sleep habits. Any such sensor and bands known to a skilled person for measuring activities, physiological functions, vital signs, behaviors, and environmental factors related to a person are within the scope of the present invention for determining parameters for multiple factors that can affect the medication dose.

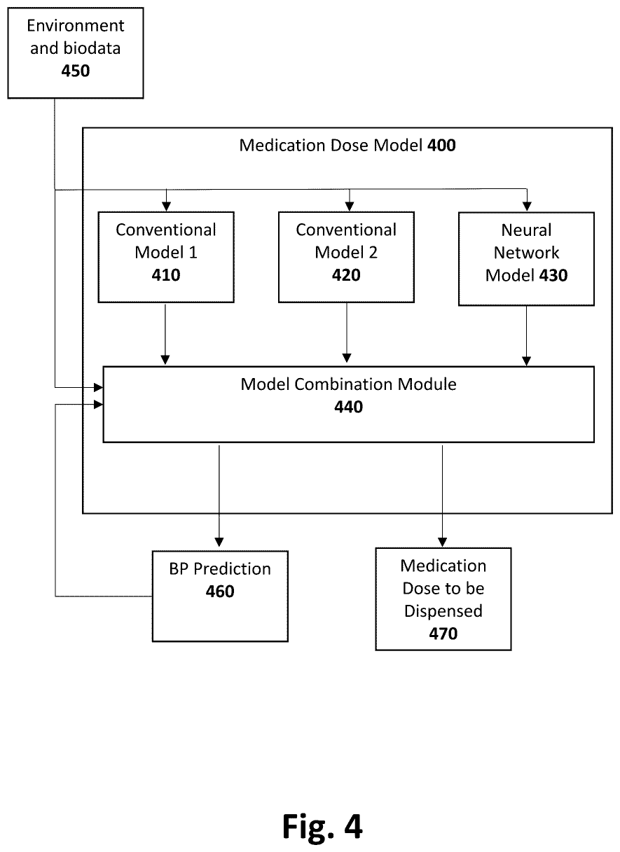

Referring to FIG. 4 which is a block diagram illustrating an exemplary embodiment of the medication dose model 400. The disclosed medication dose model 400 can be a neural-network-based model that can be complemented by other types of conventional first order and second-order models (FIG. 4). A first-order model 410 is governed by a first-order differential equation, whereas a second-order model 420 is governed by a second-order differential equation. We envision combining the first-order model 410, the second-order model 420, and the neural network model 430 into a model combination module 440 with the data 450 acquired from the real world being used to adjust the weight of each section of the algorithm. It is envisioned as a hybrid model consisting of conventional (differential equations) as well as neural network models.

The advantage of the hybrid model be that the algorithm can adjust based on accurate or estimated values of the multiple parameters. During the training phase (phase I), the model can constantly adjust its internal structure and parameters to fit the patient's blood pressure data, so that it can predict with sufficient accuracy the blood pressure. The model can take input all types of data including multiple parameters affecting the blood pressure as described above and predict blood pressure values for the patient. The predicated or observed blood pressure values can be compared with the actual blood pressure values of the patient to obtain feedback. Once the model can produce the required predictions using data it has not used for training, the model can be validated. This is where the medical professional comes in and allows the model to take over in deciding the medication dose (Phase II). The algorithm can keep receiving different parameters and blood pressure data of the patient, and the algorithm is constantly trained with new data. As such, the algorithm adapts itself to the new conditions of the patient.

In certain implementations, the disclosed apparatus may be integrated with one or more healthcare devices, such as a smartwatch, a glucose monitoring kit, a blood pressure monitoring kit, or similar devices. The apparatus may include, or be operably coupled to, various sensors configured to measure physiological parameters including, but not limited to, blood pressure, oxygen saturation, pulse rate, blood glucose levels, sleep patterns, and related health indicators. Such sensors may be integrated within the apparatus itself or may form part of a system associated with the apparatus and communicatively coupled thereto.

Accordingly, the disclosed apparatus may function as a personal healthcare assistant configured to track, monitor, and manage healthcare-related data and medication information of a user. In preferred implementations, the system may operate in an autonomous or semi-autonomous manner, requiring minimal user intervention. For example, glucose monitoring sensors may automatically measure glucose levels according to a predefined schedule without requiring manual measurement or user input. As a result, patient compliance may be significantly improved, thereby providing a substantial advancement in healthcare monitoring and management systems.

The system may include an interface module configured to receive data from a plurality of healthcare devices and one or more healthcare-related databases. The interface module may further allow entry and configuration of parameters for operably coupling the disclosed system to external devices. For example, via the interface module, the system may be configured to, according to a predefined schedule and autonomously, to read data from one or more external devices.

A wide variety of healthcare devices from different manufacturers are known in the art and may have differing setup, communication, and operational requirements. The interface module may therefore provide universal or multi-protocol compatibility, enabling the system to connect with and receive data from such heterogeneous external devices. In this manner, the system may automatically receive readings from the external devices, thereby improving convenience and enhancing patient compliance.

In certain implementations, the interface module may be updatable to support compatibility with newly introduced or updated external devices. While autonomous acquisition of data from external devices may be preferred, the system may additionally or alternatively support manual data entry by a user.

The acquisition of readings from external devices at a predefined schedule may include obtaining readings in real time or near real time. The system may connect to the external devices through one or more suitable communication networks or interfaces. In some embodiments, the system may automatically detect availability of an external device, for example by periodically polling or pinging the external device, and may establish a connection when the external device is powered on or becomes available, in order to obtain readings therefrom.

Additionally, in certain implementations, the system may incorporate machine vision technology through a connected camera and, optionally, a microphone. The system may detect, via machine vision, that a user is taking a reading using an external device and may accordingly acquire or extract the corresponding reading. In some embodiments, the system may provide a user authorization or confirmation mechanism for approving newly acquired input before such data is incorporated into further processing or analysis.

The disclosed system may further address trust, integrity, and accountability concerns by incorporating distributed ledger technologies, such as blockchain or blockchain-like systems. In certain implementations, system events including, but not limited to, data inputs, processing operations, outputs, and related transactions may be recorded, logged, or anchored to a distributed ledger.

Recording such events on the distributed ledger may provide tamper-resistance, data integrity verification, and traceability, thereby safeguarding against unauthorized modification of data and enhancing trust and accountability of the disclosed system. In some embodiments, cryptographic techniques associated with the distributed ledger may be used to validate the authenticity and immutability of recorded events.

In certain implementations, the apparatus may include dispensing units that may be configured to dispense cartridges (injectables), inhaler canisters, or transdermal patch applicators.

In certain implementations, disclosed herein is a method for determining an optimum medication dosage and for adaptively improving the dosage over time based on changing factors. The system may receive diagnostic data from one or more wearable devices, wherein the diagnostic data may be collected over a period of time and may include a plurality of readings. The system may further receive biodata associated with a user.

As used herein, the term biodata broadly refers to data relating to the user, a medical condition of the user, and an ongoing or prescribed treatment regimen. Using a reinforcement-learning-based machine learning model, the system may process the diagnostic data and the biodata to determine an optimized medication dosage and a predicted or observed patient outcome associated with the optimized dosage. The optimized dosage may be applied for a defined period of time, after which the system may reevaluate and update the optimum dosage based on newly received data.

Upon determining an optimum medication dosage, the system may present the determined dosage to a healthcare service provider via the telehealth interface. Based on predefined configuration settings, the system may dispense the determined dosage to the patient either autonomously or after receiving approval from the healthcare service provider.

In certain implementations, also disclosed herein is an apparatus configured to calculate and dispense medication dosages for a patient and to optimize the medication dosage over time based on treatment outcomes and changing physiological and environmental factors. The apparatus may include a dispensing unit configured to dispense medication in a plurality of delivery formats.

In certain implementations, the apparatus may be implemented in the form of, or incorporated into, a wearable device. By way of example, the wearable device may comprise a smartwatch including a plurality of sensors configured to monitor healthcare-related parameters. The disclosed apparatus may additionally or alternatively be operably coupled to one or more external medication delivery devices, such as a smart inhaler.

In certain implementations, the apparatus may further include a record module configured to record inputs, outputs, and operational logs of the apparatus in a tamper-resistant data store, such as a blockchain or blockchain-based distributed ledger.

In certain implementations, the dispensing unit comprises a modular cartridge system for interchangeable drug forms.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for calculating an optimum medication dosage for a medical condition in a patient, the method being implemented by an apparatus comprising a processor, a memory, one or more sensors configured to determine one or more health parameters of the patient, and a dispensing station, the method comprising:
creating and training a machine-learning-based medication dosage model using at least diagnostic data, medication data, and biodata of the patient to determine a medication dosage for the medical condition of the patient, wherein the diagnostic data and the medication data are time-stamped to form time-series data, and wherein training the machine-learning-based medication dosage model comprises:
predicting, by the machine-learning-based medication dosage model, one or more health parameters as observations;
comparing the predicted one or more health parameters with predetermined criteria or actual values of the one or more health parameters;
upon determining a match between the predicted health parameters and the predetermined criteria or actual values, storing the observations; and
upon determining a non-match, generating feedback for adjusting the machine-learning-based medication dosage model; and
storing the trained medication dosage model in the memory;
processing, by the processor, the medication dosage model based on at least current diagnostic data and current medication data of the patient to determine a first medication dosage; and
dispensing, by the dispensing station of the apparatus, the determined first medication dosage by combining sub-unit amounts of medication in one or more dosage combinations.

2. The method of claim 1, wherein the method further comprises:
transmitting, through a telehealth interface, the first medication dosage, the at least current diagnostic data, and the current medication data to a remote clinician dashboard.

3. The method of claim 1, wherein the machine-learning-based medication dosage model comprises reinforcement learning.

4. The method of claim 1, wherein the dispensing station is configured to deliver a plurality of medication delivery formats.

5. The method of claim 1, wherein the method further comprises recording the determined first medication dosage, the at least current diagnostic data, and the current medication data in a tamper-resistant database.

6. The method of claim 1, wherein the method further comprises rendering an interface for configuring one or more external medical devices to autonomously receive readings from the one or more external medical devices.

7. The method of claim 1, wherein the apparatus further comprises a device including the one or more sensors, the device comprising at least one of a glucose monitor, a smartwatch, or a smart inhaler.

8. The method of claim 1, wherein the method further comprises updating, through the telehealth interface enables, the first medication dosage with a forced medical dosage.

9. An apparatus for calculating an optimum medication dosage for a medical condition in a patient, the apparatus comprising a processor, a memory, one or more sensors configured to determine one or more health parameters of the patient, and a dispensing station, the apparatus is configured to:
process a machine-learning-based medication dosage model based on at least current diagnostic data and current medication data of the patient to determine a first medication dosage; and
dispense, by the dispensing station of the apparatus, the determined first medication dosage by combining sub-unit amounts of medication in one or more dosage combinations.
